(19) United States
(12) Patent Application Publication
(10) Pub. No.: US 2005/0228052 A1
(43) Pub. Date: Oct. 13, 2005

(54) Methods of treating, preventing and managing a sleep disorder using (S)-didesmethylsibutramine

(76) Inventor: Timothy J. Barberich, Concord, MA (US)

Correspondence Address: JONES DAY, 51 Louisiana Aveue, N.W, WASHINGTON, DC, 20001-2113, US

(21) Appl. No.: 11/044,566

(22) Filed: Jan. 28, 2005

Related U.S. Application Data
(60) Provisional application No. 60/539,743, filed on Jan. 29, 2004.

(51) Int. Cl. A61K 31/137

(52) U.S. Cl. 514/650

(57) ABSTRACT

This invention relates, in part, to methods of treating, preventing and/or managing a sleep disorder using enantiomerically pure (S)-didesmethylsibutramine, or a pharmaceutically acceptable salt, hydrate, solvate, clathrate or prodrug thereof. Specific methods for treating, preventing and/or managing insomnia, wakefulness, circadian rhythm sleep disorders, shift work sleep disorder, and periodic limb movement disorder are also disclosed.

This application claims priority to U.S. Provisional Patent No. 60/539,743, filed Jan. 29, 2004, which is incorporated herein in its entirety by reference.

### 1. FIELD OF THE INVENTION

This invention relates to methods of treating, preventing and managing a sleep disorder.

### 2. BACKGROUND OF THE INVENTION

Sleep is a complicated process involving many different parts of the nervous system. The mechanism that induces sleep is not known, nor is why exactly sleep is necessary for good health and efficient mental functioning. The Merck Manual, 17^{th} Ed., p. 1409 (Merck Research Laboratories, White House Station, N.J., 1999), page 1409.

Sleep consists of two very different stages: rapid eye movement sleep (REM) and non-rapid eye movement sleep (NREM). In REM sleep, the eyes move under the closed lids, and body processes speed up. Periods of REM sleep typically last for about 20 minutes, and occur 4 to 5 times during the night. During a normal night's sleep, REM sleep follows each of 4 to 6 cycles of NREM sleep. Id. NREM sleep is typically classified into four stages. McGraw-Hill Concise Encyclopedia of Science & Technology, 3^{rd} Ed. (McGraw-Hill, Inc., 1994), page 1710. A normal night of sleep for a young adult typically consists of: about 50% in stage 2 sleep; about 20% in stages 3 and 4 sleep; about 25% in REM sleep; and about 5% in stage 1 sleep. Id.

For most people, falling and staying asleep, or waking and staying awake, are natural processes. For people with sleep disorders, however, problems falling and staying asleep, or waking and staying awake, persist, and can impair their daily routines.

The International Classification of Sleep Disorders (ICSD) lists over seventy sleep disorders. The International Classification of Sleep Disorders, Revised: Diagnostic and Coding Manual (ICSD-R), American Academy of Sleep Medicine (2001). Broadly, the symptoms associated with these sleep disorders include: problems falling asleep and staying asleep; problems staying awake; difficulties staying with a regular sleep/awake cycle; repetitive limb movements; sleepwalking; bedwetting; nightmares; and other problems that interfere with sleep. Sleep disorders can lead to lowered quality of life and reduced personal health. They also endanger public safety by contributing to a number of traffic and industrial accidents.

2.1 Insomnia

Insomnia refers to difficulty in falling asleep or in staying asleep or disturbed sleep patterns resulting in insufficient sleep, and is the most common sleep disorder. It varies from restless or disturbed sleep, to a reduction in the usual time spent sleeping. In extreme cases, insomnia can involve complete wakefulness.

Insomnia is a common syndrome: about 10 percent of the population have chronic insomnia and about 50 percent have significant insomnia at some time. Insomnia can be further categorized into primary and secondary insomnia. Primary insomnia refers to long-standing insomnia with little or no relationship to immediate or somatic or psychic events. Secondary insomnia refers to insomnia secondary to emotional problems, pain, physical disorders or use or withdrawal of drugs. The Merck Manual, 17^{th} Ed., page 1410.

Initial insomnia refers to difficulty in falling asleep. Initial insomnia is commonly associated with an emotional disturbance such as anxiety, a phobic state or depression. In some cases, it is also associated with pain, respiratory problems, stimulant drugs, withdrawal of sedative drugs and/or poor sleep hygiene (e.g., a variable sleep schedule). Initial insomnia can also be associated with other sleep disorders such as restless leg syndrome and sleep apnea.

Middle insomnia refers to difficulty in remaining asleep during the night. Rebound wakefulness, one form of middle insomnia, commonly occurs when hypnotics are withdrawn from a patient who regularly takes heavy doses.

Terminal insomnia refers to waking up too early. It is also referred to as early morning awakening syndrome, in which a patient falls asleep normally but awakens early and cannot fall asleep again or drifts into a restless, unsatisfying sleep. This pattern is a common phenomenon of aging but can also be associated with depression. It has been reported that tendencies to anxiety, self-reproach and self-punitive thinking, often magnified in the morning, may contribute to the disorder.

2.2 Circadian Rhythm Sleep Disorders

Circadian rhythm sleep disorders refer to irregularities in sleep caused by circadian rhythm misalignment.

Delayed sleep phase syndrome is a circadian rhythm disturbance in which a patient has delayed sleep and waking times and cannot advance her sleep schedule, i.e., cannot move to an earlier bedtime with an earlier awakening time.

Shift Work Sleep Disorder (“SWSD”), also referred to as shift work mal-adaptation, is the most common condition caused by circadian rhythm misalignment. SWSD consists of symptoms of insomnia or excessive sleepiness that occur as transient phenomena in relation to work schedules.

Sleep rhythm reversals usually reflect a circadian rhythm disorder or damage to the hypothalamic region of the diencephalon. Other causes of sleep rhythm reversals include misuse of sedatives, working irregular shift and obstructive sleep apnea.

Time-zone change syndrome, also referred to as jetlag or circadian dysrhythmia, is typically caused when rapid travel across multiple time zone disrupts the normal circadian rhythm. The Merck Manual, 17^{th} Ed., page 2457.

2.3 Periodic Limb Movement Disorder

Periodic limb movements in sleep (PLMS), periodic limb movement disorder (PLMD) or nocturnal myoclonus are sleep disorders that involve involuntary (not consciously controlled) periodic episodes of repetitive limb movements during sleep that occur about every 20-40 seconds. The limb movements typically occur in the lower limbs or legs, but may occasionally also affect the arms, and can include without limitation, brief muscle twitches, jerking movements, or an upward flexing of the feet. Typically, the limb movements do not occur throughout the night or sleep cycle, but instead cluster in first portion of sleep or during non-REM sleep. The limb movements are much less common during REM sleep because the muscles are normally paralyzed during these phase of sleep to prevent a person from physically acting out their dreams.

PLMS or PLMD can result in a patient having various complaints about sleep, including without limitation, difficulty falling asleep, trouble in staying asleep or going back to sleep once they've awakened, or excessive daytime sleepiness. In many cases, the patient themselves may not report any difficulty with sleep, but their bed partner will report being disturbed by the movements, such as complaining of being hit or kicked by the patient during the night. The varied complaints about sleep that patients can have with PLMS or PLMD all arise from the same cause, but involve differences in the patients' timing and perception of the problem. For example, some patients may not be consciously aware of any sleep disturbance, but the many microarousals or brief awakenings during the night do disturb sleep and cause excessive daytime sleepiness. In other situations, limb movements occurring immediately after a patient falls asleep may awaken them before they realize they have fallen asleep, leading the patient to perceive that they have difficulty falling asleep.

2.4 Methods of Treatment

Certain sleep disorders can be treated with drugs. Sleeping pills and drugs that promote alertness are among those most commonly used. Typical sleeping pills include hypnotics, sedatives, anxiolytics, GABA enhancers, antihistamines, antidepressants, neuroleptics, dopaminergic agents and opioids. However, all of these drugs have various limitations, such as quick development of tolerance, severe side effects, and in extreme cases, development of addition and dependency. CNS stimulants, which are commonly used to induce alertness, are also associated with severe side effects. So, too are tricyclic antidepressants and serotonin reuptake inhibitors, which are used to induce alertness. Therefore, a need exists for a drug that can be safely and effectively used in treating, preventing or managing sleeping disorders.

### 3. SUMMARY OF THE INVENTION

This invention is directed, in part, to a method of treating, preventing or managing a sleep disorder comprising administering to a patient in need of such treatment, prevention or management a therapeutically effective amount of enantiomerically pure (S)-didesmethylsibutramine, or a pharmaceutically acceptable salt, hydrate, solvate, clathrate or prodrug thereof.

Examples of sleep disorders include, but are not limited to, circadian rhythm sleep disorders (e.g., shift work sleep disorder), insomnia (e.g., complete wakefulness), and periodic limb movements in sleep, periodic limb movement disorder or nocturnal myoclonus.

In one embodiment, (S)-didesmethylsibutramine comprises greater than about 90 percent, greater than about 95 percent, greater than about 97 percent, or greater than about 99 percent by weight of the didesmethylsibutramine administered to a patient.

In another embodiment, (S)-didesmethylsibutramine is administered in an amount of from about 0.1 mg to about 60 mg per day. In a specific embodiment, (S)-didesmethylsibutramine is administered in an amount of from about 2 mg to about 30 mg per day, and more specifically from about 5 mg to about 15 mg per day.

In another embodiment, (S)-didesmethylsibutramine is administered orally, mucosally, rectally, transdermally, or parenterally. Examples of parenteral administration include, but are not limited to, intravenous, intramuscular and subcutaneous administration.

### 4. DETAILED DESCRIPTION OF THE INVENTION

This invention is based, in part, on a realization that enantiomerically pure (S)-didesmethylsibutramine, or a pharmaceutically acceptable salt, hydrate, solvate, clathrate or prodrug thereof, can be used to treat, prevent or manage various diseases or disorders. (S)-didesmethylsibutramine, which is chemically named 1-[1-(4-chlorophenyl)cyclobutyl]-3-methyl-butylamine, has the structure shown below:

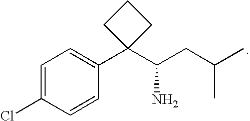

As used herein, the term “pharmaceutically acceptable salt” refers to salts prepared from pharmaceutically acceptable non-toxic acids, including inorganic acids and organic acids. Suitable non-toxic acids include inorganic and organic acids such as, but not limited to, acetic, alginic, anthranilic, benzenesulfonic, benzoic, camphorsulfonic, citric, ethenesulfonic, formic, fumaric, furoic, gluconic, glutamic, glucorenic, galacturonic, glycidic, hydrobromic, hydrochloric, isethionic, lactic, maleic, malic, mandelic, methanesulfonic, mucic, nitric, pamoic, pantothenic, phenylacetic, propionic, phosphoric, salicylic, stearic, succinic, sulfanilic, sulfuric, tartaric acid, p-toluenesulfonic and the like. Particularly preferred are hydrochloric, hydrobromic, phosphoric, and sulfuric acids, and most particularly preferred is the hydrochloride salt.

As used herein, and unless otherwise specified, the term “prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise —NO, —NO₂, —ONO, or —ONO₂ moieties. The term “prodrug” is accorded a meaning herein such that prodrugs of (S)-didesmethylsibutramine do not encompass (S)-sibutramine or (S)-desmethylsibutramine.

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

4.1 Methods of Treatment, Prevention or Management

This invention is directed, in part, to a method of treating, preventing or managing a sleep disorder comprising administering to a patient in need of such treatment, prevention or management a therapeutically or prophylactically effective amount of (S)-didesmethylsibutramine, or a pharmaceutically acceptable salt, solvate, hydrate, clathrate or prodrug thereof.

As used herein, and unless otherwise specified, the terms “treat,” “treating” and “treatment” refer to the eradication or amelioration of a disease or condition, or of one or more symptoms associated with the disease or condition. In certain embodiments, the terms refer to minimizing the spread or worsening of the disease or condition resulting from the administration of one or more prophylactic or therapeutic agents to a subject with such a disease or condition.

As used herein, and unless otherwise specified, the terms “prevent,” “preventing” and “prevention” refer to the prevention of the onset, recurrence or spread of a disease or condition, or of a symptom thereof.

As used herein, and unless otherwise specified, the terms “manage,” “managing” and “management” refer to preventing or slowing the progression, spread or worsening of a disease or condition, or of a symptom thereof. Often, the beneficial effects that a subject derives from a prophylactic or therapeutic agent do not result in a cure of the disease or condition.

As used herein, and unless otherwise specified, the term “sleep disorder” refers to a disorder that manifests symptoms which include abnormal sleep cycles, e.g., difficulty in falling and staying asleep, difficulty in staying awake, sleep fragmentation, irregularities in sleep/wake cycle, and excessive day time sleepiness. Specific examples of sleep disorders include, but are not limited to, those listed in ICSD-R (2001), the entirety of which is incorporated herein by reference, and those listed below.

As used herein, and unless otherwise specified, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease or condition, or one or more symptoms associated with the disease or condition, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “enantiomerically pure” means a composition that comprises one enantiomer of a compound and is substantially free of the opposite enantiomer of the compound. A typical enantiomerically pure compound comprises greater than 90 percent by weight of one enantiomer of the compound and less than about 10 percent by weight of the opposite enantiomer of the compound, preferably greater than about 95 percent by weight of one enantiomer of the compound and less than about 5 percent by weight of the opposite enantiomer of the compound, and more preferably greater than about 97 percent by weight of one enantiomer of the compound and less than about 3 percent by weight of the opposite enantiomer of the compound, and even more preferably greater than about 99 percent by weight of one enantiomer of the compound and less than about 1 percent by weight of the opposite enantiomer of the compound. For example, enantiomerically pure (S)-didesmethylsibutramine in one embodiment comprises at least about 90 percent by weight (R)-didesmethylsibutramine and less than about 10 percent by weight (S)-didesmethylsibutramine.

In the methods of the invention, a therapeutically or prophylactically effective amount of (S)-didesmethylsibutramine, or a pharmaceutically acceptable salt, solvate, hydrate, clathrate or prodrug thereof, is administered to a patient. In a specific embodiment, the patient is a mammal such as a human, a dog or a cat, preferably a human.

In specific methods of the invention, (S)-didesmethylsibutramine, or a pharmaceutically acceptable salt, solvate, hydrate, clathrate or prodrug thereof, is administered to a patient in an amount from about 0.1 mg to about 60 mg, from about 2 mg to about 30 mg, from about 5 mg to about 15 mg. Such amounts can be administered daily as needed for the treatment, prevention or management of acute and chronic diseases and conditions.

Optionally, enantiomerically pure (S)-didesmethylsibutramine is adjunctively administered (i.e., administered in combination) with one or more additional pharmacologically active compounds. In other words, (S)-didesmethylsibutramine and an additional pharmacologically active compound can be administered to a patient as a combination, concurrently but separately, or sequentially by any suitable route. Suitable routes of administration include oral, mucosal (e.g., nasal, sublingual, buccal, rectal, and vaginal), parenteral (e.g., intravenous, intramuscular or subcutaneous), and transdermal routes.

As physicians and those skilled in the art of pharmacology will readily appreciate, the particular additional pharmacologically active compounds that can be administered in combination with enantiomerically pure (S)-didesmethylsibutramine will depend on the particular disease or condition being treated or prevented, and may also depend on the age and health of the patient to which the compounds are to be administered.

Additional pharmacologically active compounds that can be used in the methods and compositions of the invention include drugs that act on the central nervous system (“CNS”), such as, but not limited to: 5-HT (e.g., 5-HT₃ and 5-HT_{1A}) agonists and antagonists; selective serotonin reuptake inhibitors (“SSRIs”); hypnotics and sedatives; drugs useful in treating psychiatric disorders including antipsychotic and neuroleptic drugs, antianxiety drugs, anti-anxiolytic agents, antidepressants, β-adrenergic antagonists and mood-stabilizers; CNS stimulants such as amphetamines; and dopamine receptor agonists.

The clinician, physician, or psychiatrist will appreciate which of the above compounds can be used in combination with (S)-didesmethylsibutramine, or a pharmaceutically acceptable salt, solvate, hydrate, clathrate or prodrug thereof, for the treatment, prevention or management of a given disorder.

Enantiomerically pure (S)-didesmethylsibutramine can be effectively used in treating, preventing or managing a wide variety of sleep disorders.

Sleep disorders that can be treated, prevented or managed using the compounds of this invention include, but are not limited to, those listed in ICSD Manual (2001), the entirety of which is incorporated herein by reference. Specific examples of sleep disorders that can be treated, prevented or managed using the compounds of this invention include, but are not limited to, dyssomnias and parasomnias.

Examples of dyssomnias include, but are not limited to: circadian rhythm sleep disorders such as advanced sleep-phase syndrome, delayed sleep phase syndrome, irregular sleep/wake pattern, non-24-hour sleep/wake disorder, shift-work sleep disorder, sleep rhythm reversals, time-zone change syndrome and other circadian rhythm sleep disorders known in the art; extrinsic sleep disorders such as adjustment sleep disorder, alcohol-dependent sleep disorder, altitude insomnia, environmental sleep disorder, inadequate sleep hygiene, insufficient sleep syndrome, limit-setting sleep disorder, sleep-onset association disorder, stimulant dependent sleep disorder, toxin-induced sleep disorder and other extrinsic sleep disorders known in the art; and intrinsic sleep disorders such as central alveolar hypoventilation, idiopathic insomnia, narcolepsy, obstructive sleep apnea syndrome, periodic limb movement disorder, posttraumatic hypersomnia, psychophysiological insomnia, recurrent hypersomnia, sleep state misperception and other intrinsic sleep disorders known in the art. In a specific embodiment, the sleep disorder is not restless leg syndrome.

In one embodiment, the sleep disorder to be treated, prevented or managed using the compounds of this invention is an insomnia. Examples of insomnias include, but are not limited to, primary insomnia, secondary insomnia, transient insomnia, chronic insomnia, initial insomnia, middle insomnia, and terminal insomnia. Other types of insomnia, regardless of symptoms or causes associated therewith, can be effectively treated, prevented or managed using the compounds of this invention.

In a specific embodiment, the insomnia is complete wakefulness. As used herein, the term “wakefulness” refers to a temporary state in which one is unable to sleep.

In another embodiment, the sleep disorder to be treated, prevented or managed using the compounds of this invention is a circadian rhythm sleep disorder. Examples of circadian sleep disorders include, but are not limited to, advanced sleep-phase syndrome, delayed sleep phase syndrome, irregular sleep/wake pattern, non-24-hour sleep/wake disorder, shift-work sleep disorder, sleep rhythm reversals and time-zone change syndrome.

Other circadian rhythm sleep disorders, regardless of symptoms or causes associated therewith, can be effectively treated, prevented or managed using the compounds of this invention.

In another embodiment, the circadian rhythm sleep disorder is shift-work sleep disorder. Without being limited by a particular theory, it is generally believed that shift-work sleep disorder is triggered by the misalignment between the external sleep-wake patterns and the internal sleep-wake processes. In other words, excessive sleepiness is caused because the patient attempts to work when the internal sleep-wake processes are promoting sleep. Conversely, insomnia is caused because the patient attempts to sleep when the internal sleep-wake processes are promoting wakefulness. As used herein, a “shift worker” refers to its generally accepted meaning, e.g., someone who works outside the standard hours of 7 AM to 6 PM. See, e.g., Monk et al., Making Shift Work Tolerable (Taylor and Francis, Inc., London, U.K. and Washington, D.C., 1992).

Examples of parasomnias include, but are not limited to: parasomnias associated with REM sleep such as impaired sleep-related penile erections, nightmares sleep paralysis, REM sleep behavior disorder, REM sleep-related sinus arrest and sleep-related painful erections; sleep/wake transition disorders such as arousal disorders, night terrors (payor nocturnus or incubus attacks), rhythmic movement disorder, periodic limb movement in sleep (PLMS), periodic limb movement disorder (PLMD), nocturnal myoclonus, sleep starts (hypnic jerks), sleep talking and sleepwalking (somnambulism); and other parasomnias such as benign neonatal sleep myoclonus, congenital central hypoventilation syndrome, nocturnal paroxysmal dystonia, primary snoring infant sleep apnea, sleep bruxism (teeth grinding), sleep enuresis (bed wetting), sleep-related abnormal swallowing syndrome, sudden infant death syndrome, sudden unexplained nocturnal death syndrome and other parasomnias known in the art. In a specific embodiment, the sleep disorder is not sleep apnea.

In one embodiment, the parasomnia sleep disorder to be treated, prevented or managed using (S)-didesmethylsibutramine, or a pharmaceutically acceptable salt, hydrate, solvate, clathrate, or prodrug thereof, is periodic limb movement in sleep, periodic limb movement disorder, or nocturnal myoclonus.

### 4.2 Synthesis of (S)-DIDESMETHYLSIBUTRAMINE

Racemic didesmethylsibutramine can be prepared by methods known to those of ordinary skill in the art. See, e.g., U.S. Pat. No. 4,806,570, which is incorporated herein by reference; J. Med. Chem., 2540 (1993) (tosylation and azide replacement); Butler, D., J. Org. Chem., 36:1308 (1971) (cycloalkylation in DMSO); Tetrahedron Lett., 155-58 (1980) (Grignard addition to nitrite in benzene); Tetrahedron Lett., 857 (1997) (OH to azide); and Jeffery, J. E., et al., J. Chem. Soc. Perkin. Trans 1, 2583 (1996).

Racemic didesmethylsibutramine can be prepared from racemic sibutramine or desmethylsibutramine, as can optically pure forms of the compound. Optically pure enantiomers of didesmethylsibutramine can be prepared using techniques known in the art. A preferred technique is resolution by fractional crystallization of diastereomeric salts formed with optically active resolving agents. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, NY, 1962); and S. H. Wilen Tables of Resolving Agents and Optical Resolutions 268 (E. L. Eliel ed., Univ. of Notre Dame Press, Notre Dame, Ind., 1972).

Because didesmethylsibutramine is a basic amine, diastereomeric salts of the compound that are suitable for separation by fractional crystallization are readily formed by addition of optically pure chiral acid resolving agents. Suitable resolving agents include, but are not limited to, optically pure tartaric, camphorsulfonic acid, mandelic acid, and derivatives thereof. Optically pure isomers of didesmethylsibutramine can be recovered either from the crystallized diastereomer or from the mother liquor, depending on the solubility properties of the particular acid resolving agent employed and the particular acid enantiomer used. The identity and optical purity of the particular didesmethylsibutramine so recovered can be determined by polarimetry or other analytical methods.

Racemic and optically pure didesmethylsibutramine are preferably synthesized directly by methods such as those disclosed by Jeffery, J. E., et al., J. Chem. Soc. Perkin. Trans 1, 2583 (1996).

A preferred method of directly synthesizing racemic didesmethylsibutramine comprises the reaction of CCBC with a compound of formula i-BuMX, wherein X is Br or I and M is selected from the group consisting of Li, Mg, Zn, Cr, and Mn. Preferably, the compound is of the formula i-BuMgBr. The product of this reaction is then reduced under suitable reaction conditions.

The enantiomers of didesmethylsibutramine can be resolved by the formation of chiral salts, as described above. Preferred chiral acids used to form the chiral salts include, but are not limited to, tartaric acid. Preferred solvent systems include, but are not limited to, acetonitrile/water/methanol and acetonitrile/methanol.

4.3 Pharmaceutical Compositions

This invention encompasses pharmaceutical compositions comprising enantiomerically pure (S)-didesmethylsibutramine, or a pharmaceutically acceptable salt, hydrate, solvate, clathrate, or prodrug thereof. Certain pharmaceutical compositions are single unit dosage forms suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic or hard gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the compounds of this invention from degradation within the gastrointestinal tract. In another example, the compounds of this invention may be administered in a liposomal formulation to shield the compounds from degradative enzymes, facilitate transport in circulatory system, and effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

The selected dosage level and frequency of administration of the pharmaceutical compositions of the invention will depend upon a variety of factors including the route of administration, the time of administration, the rate of excretion of the therapeutic agents, the duration of the treatment, other drugs, compounds and/or materials used in the patient, the age, sex, weight, condition, general health and prior medical history of the patient being treated, and like factors well known in the medical arts. For example, the dosage regimen is likely to vary with pregnant women, nursing mothers and children relative to healthy adults. A physician having ordinary skill in the art can readily determine and prescribe the therapeutically effective amount of the pharmaceutical composition required.

The pharmaceutical compositions of the invention comprising (S)-didesmethylsibutramine, or a pharmaceutically acceptable salt, hydrate, solvate, clathrate, or prodrug thereof, may further comprise a pharmaceutically acceptable carrier. The term “pharmaceutically acceptable carrier” means one or more pharmaceutically acceptable excipients. Examples of such excipients are well known in the art and are listed in the USP (XXI)/NF (XVI), incorporated herein in its entirety by reference thereto, and include without limitation, binders, diluents, fillers, disintegrants, super disintegrants, lubricants, surfactants, antiadherents, stabilizers, and the like. The term “additives” is synonymous with the term “excipients” as used herein.

The term “pharmaceutically acceptable” is used herein to refer to those compounds, materials, compositions and/or dosage forms which are, within the scope of sound medical judgment, suitable for administration to and for use in contact with the tissues and fluids of human beings and animals without excessive toxicity, irritation, allergic response, or other problem or complication, commensurate with a reasonable medically sound benefit/risk ratio.

Further, the term “pharmaceutically acceptable” excipient is employed to mean that there are no untoward chemical or physical incompatibilities between the active ingredients and any of the excipient components of a given dosage form. For example, an untoward chemical reaction is one wherein the potency of (S)-didesmethylsibutramine is detrimentally reduced or increased due to the addition of one or more excipients. Another example of an untoward chemical reaction is one wherein the taste of the dosage form becomes excessively sweet, sour or the like to the extent that the dosage form becomes unpalatable. Each excipient must be “acceptable” in the sense of being compatible with the other ingredients of the formulation and not injurious to the patient.

Physical incompatibility refers to incompatibility among the various components of the dosage form and any excipient(s) thereof. For example, the combination of the excipient(s) and the active ingredient(s) may form an excessively hygroscopic mixture or an excessively segregated mixture to the degree that the desired shape of the dosage form (e.g., tablet, troche etc.), its stability or the like cannot be sufficiently maintained to be able to administer the dosage form in compliance with a prescribed dosage regimen as desired.

It is noted that all excipients used in the pharmaceutical compositions or dosage forms made in accordance with the present invention preferably meet or exceed the standards for pharmaceutical ingredients and combinations thereof in the USP/NF. The purpose of the USP/NF is to provide authoritative standards and specifications for materials and substances and their preparations that are used in the practice of the healing arts. The USP/NF establish titles, definitions, descriptions, and standards for identity, quality, strength, purity, packaging and labeling, and also, where practicable provide bioavailability, stability, procedures for proper handling and storage and methods for their examination and formulas for their manufacture or preparation.

The stability of a pharmaceutical product may be defined as the capability of a particular formulation, in a specific container, to remain within its physical, chemical, microbiological, therapeutic and toxicological specification, although there are exceptions, and to maintain at least about 90% of labeled potency level. Thus, for example, expiration dating is defined as the time in which the pharmaceutical product will remain stable when stored under recommended conditions.

Many factors affect the stability of a pharmaceutical product, including the stability of the therapeutic ingredient(s), the potential interaction between therapeutic and inactive ingredients and the like. Physical factors such as heat, light and moisture may initiate or accelerate chemical reactions.

4.3.1 Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

Large-scale production of pharmaceutical compositions or dosage forms in accordance with the present invention may require, in addition to the therapeutic drug ingredients, excipients or additives including, but not limited to, diluents, binders, lubricants, disintegrants, colorants, flavors, sweetening agents and the like or mixtures thereof. By the incorporation of these and other additives, a variety of dosage forms (e.g., tablets, capsules, caplets, troches and the like) may be made. These include, for example, hard gelatin capsules, caplets, sugar-coated tablets, enteric-coated tablets to delay action, multiple compressed tablets, prolonged-action tablets, tablets for solution, effervescent tablets, buccal and sublingual tablets, troches and the like.

Hence, unit dose forms or dosage formulations of a pharmaceutical composition of the present invention, such as a troche, a tablet or a capsule, may be formed by combining a desired amount of each of the active ingredients with one or more pharmaceutically compatible or acceptable excipients, as described below, in pharmaceutically compatible amounts to yield a unit dose dosage formulation the desired amount of each active ingredient. The dose form or dosage formulation may be formed by methods well known in the art.

Tablets are often a preferred dosage form because of the advantages afforded both to the patient (e.g., accuracy of dosage, compactness, portability, blandness of taste as well as ease of administration) and to the manufacturer (e.g., simplicity and economy of preparation, stability as well as convenience in packaging, shipping and dispensing). Tablets are solid pharmaceutical dosage forms containing therapeutic drug substances with or without suitable additives.

Tablets are typically made by molding, by compression or by generally accepted tablet forming methods. Accordingly, compressed tablets are usually prepared by large-scale production methods while molded tablets often involve small-scale operations. For example, there are three general methods of tablet preparation: (1) the wet-granulation method; (2) the dry-granulation method; and (3) direct compression. These methods are well known to those skilled in the art. See Remington's Pharmaceutical Sciences, 16th and 18th Eds., Mack Publishing Co., Easton, Pa. (1980 and 1990). See also U.S. Pharmacopeia XXI, U.S. Pharmacopeial Convention, Inc., Rockville, Md. (1985).

Various tablet formulations may be made in accordance with the present invention. These include tablet dosage forms such as sugar-coated tablets, film-coated tablets, enteric-coated tablets, multiple-compressed tablets, prolonged action tablets and the like. Sugar-coated tablets (SCT) are compressed tablets containing a sugar coating. Such coatings may be colored and are beneficial in covering up drug substances possessing objectionable tastes or odors and in protecting materials sensitive to oxidation. Film-coated tablets (FCT) are compressed tablets that are covered with a thin layer or film of a water-soluble material. A number of polymeric substances with film-forming properties may be used. The film coating imparts the same general characteristics as sugar coating with the added advantage of a greatly reduced time period required for the coating operation. Enteric-coated tablets are also suitable for use in the present invention. Enteric-coated tablets (ECT) are compressed tablets coated with substances that resist dissolution in gastric fluid but disintegrate in the intestine. Enteric coating can be used for tablets containing drug substances that are inactivated or destroyed in the stomach, for those which irritate the mucosa or as a means of delayed release of the medication.

Multiple compressed tablets (MCT) are compressed tablets made by more than one compression cycle, such as layered tablets or press-coated tablets. Layered tablets are prepared by compressing additional tablet granulation on a previously compressed granulation. The operation may be repeated to produce multilayered tablets of two, three or more layers. Typically, special tablet presses are required to make layered tablets. See, for example, U.S. Pat. No. 5,213,738, incorporated herein in its entirety by reference thereto.

Press coated tablets are another form of multiple compressed tablets. Such tablets, also referred to as dry-coated tablets, are prepared by feeding previously compressed tablets into a tableting machine and compressing another granulation layer around the preformed tablets. These tablets have all the advantages of compressed tablets, i.e., slotting, monogramming, speed of disintegration, etc., while retaining the attributes of sugar coated tablets in masking the taste of the drug substance in the core tablet. Press-coated tablets can also be used to separate incompatible drug substances. Further, they can be used to provide an enteric coating to the core tablets. Both types of tablets (i.e., layered tablets and press-coated tablets) may be used, for example, in the design of prolonged-action dosage forms of the present invention.

Pharmaceutical compositions or unit dosage forms of the present invention in the form of prolonged-action tablets may comprise compressed tablets formulated to release the drug substance in a manner to provide medication over a period of time. There are a number of tablet types that include delayed-action tablets in which the release of the drug substance is prevented for an interval of time after administration or until certain physiological conditions exist. Repeat action tablets may be formed that periodically release a complete dose of the drug substance to the gastrointestinal fluids. Also, extended release tablets that continuously release increments of the contained drug substance to the gastrointestinal fluids may be formed.

In order for medicinal substances or therapeutic ingredients of the present invention, with or without excipients, to be made into solid dosage forms (e.g., tablets) with pressure, using available equipment, it is necessary that the material, either in crystalline or powdered form, possess a number of physical characteristics. These characteristics can include, for example, the ability to flow freely, as a powder to cohere upon compaction, and to be easily released from tooling. Since most materials have none or only some of these properties, methods of tablet formulation and preparation have been developed to impart these desirable characteristics to the material which is to be compressed into a tablet or similar dosage form.

As noted, in addition to the drugs or therapeutic ingredients, tablets and similar dosage forms may contain a number of materials referred to as excipients or additives. These additives are classified according to the role they play in the formulation of the dosage form such as a tablet, a caplet, a capsule, a troche or the like. One group of additives include, but are not limited to, binders, diluents (fillers), disintegrants, lubricants, and surfactants. In one embodiment the diluent, binder, disintegrant, and lubricant are not the same.

A binder is used to provide a free-flowing powder from the mix of tablet ingredients so that the material will flow when used on a tablet machine. The binder also provides a cohesiveness to the tablet. Too little binder will give flow problems and yield tablets that do not maintain their integrity, while too much can adversely affect the release (dissolution rate) of the drugs or active ingredients from the tablet. Thus, a sufficient amount of binder should be incorporated into the tablet to provide a free-flowing mix of the tablet ingredients without adversely affecting the dissolution rate of the drug ingredients from the tablet. With lower dose tablets, the need for good compressibility can be eliminated to a certain extent by the use of suitable diluting excipients called compression aids. The amount of binder used varies upon the type of formulation and mode of administration, and is readily discernible to those of ordinary skill in the art.

Binders suitable for use with dosage formulations made in accordance with the present invention include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone (povidone), methyl cellulose, pre-gelatinized starch, hydroxypropyl methyl cellulose, (e.g., Nos. 2208, 2906, 2910), microcrystalline cellulose or mixtures thereof. Suitable forms of microcrystalline cellulose can include, for example, the materials sold as AVICEL-PH-101, AVICEL-PH-103 and AVICEL-PH-105 (available from FMC Corporation, American Viscose Division, Avicel Sales, Marcus Hook, Pa., U.S.A.).

Fillers or diluents are used to give the powder (e.g., in the tablet or capsule) bulk so that an acceptable size tablet, capsule or other desirable dosage form is produced. Typically, therapeutic ingredients are formed in a convenient dosage form of suitable size by the incorporation of a diluent therewith. As with the binder, binding of the drug(s) to the filler may occur and affect bioavailability. Consequently, a sufficient amount of filler should be used to achieve a desired dilution ratio without detrimentally affecting release of the drug ingredients from the dosage form containing the filler. Further, a filler that is physically and chemically compatible with the therapeutic ingredient(s) of the dosage form should be used. The amount of filler used varies upon the type of formulation and mode of administration, and is readily discernible to those of ordinary skill in the art. Examples of fillers include, but are not limited to, lactose, glucose, sucrose, fructose, talc, calcium carbonate (e.g., granules or powder), microcrystalline cellulose, powdered cellulose, dextrates, kaolin, mannitol, silicic acid, sorbitol, starch, pre-gelatinized starch, or mixtures thereof.

Disintegrants are used to cause the dose form (e.g., tablet) to disintegrate when exposed to an aqueous environment. Too much of a disintegrant will produce tablets which may disintegrate in the bottle due to atmospheric moisture. Too little may be insufficient for disintegration to occur and may thus alter the rate and extent of release of drug(s) or active ingredient(s) from the dosage form. Thus, a sufficient amount of disintegrant that is neither too little nor too much to detrimentally alter the release of the drug ingredients should be used to form the dosage forms made according to the present invention. The amount of disintegrant used varies based upon the type of formulation and mode of administration, and is readily discernible to the skilled artisan. Examples of disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof.

When a dose form that dissolves fairly rapidly upon administration to the subject, e.g., in the subject's stomach is desired, a super disintegrant can be used, such as, but not limited to, croscarmellose sodium or sodium starch glycolate. The term “super disintegrant,” as used herein, means a disintegrant that results in rapid disintegration of drug or active ingredient in the stomach after oral administration. Use of a super disintegrant can facilitate the rapid absorption of drug or active ingredient(s) which may result in a more rapid onset of action.

Adhesion of the dosage form ingredients to the punches of the manufacturing machine (e.g., a tableting machine) must be avoided. For example, when drug (e.g., (S)-DDMS) accumulates on the punch surfaces, it causes the tablet surface to become pitted and therefore unacceptable. Also, sticking of drug or excipients in this way requires unnecessarily high ejection forces when removing the tablet from the die. Excessive ejection forces may lead to a high breakage rate and increase the cost of production not to mention excessive wear and tear on the dies. In practice, it is possible to reduce sticking by wet-massing or by the use of lubricants, e.g., magnesium stearate. However, selection of a drug salt with good anti-adhesion properties can also minimize these problems.

As noted, the lubricant is used to enhance the flow of the tableting powder mix to the tablet machine and to prevent sticking of the tablet in the die after the tablet is compressed. Too little lubricant will not permit satisfactory tablets to be made and too much may produce a tablet with a water-impervious hydrophobic coating, which can form because lubricants are usually hydrophobic materials such as stearic acid, magnesium stearate, calcium stearate and the like. Further, a water-impervious hydrophobic coating can inhibit disintegration of the tablet and dissolution of the drug ingredient(s). Thus, a sufficient amount of lubricant should be used that readily allows release of the compressed tablet from the die without forming a water-impervious hydrophobic coating that detrimentally interferes with the desired disintegration and/or dissolution of the drug ingredient(s).

Example of suitable lubricants for use with the present invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, or mixtures thereof. Additional lubricants include, for example, a syloid silica gel (AEROSIL 200, manufactured by W.R. Grace Co. of Baltimore Md.), a coagulated aerosol of synthetic silica (marketed by Deaussa Co. of Plano, Tex.), CAB-O-SIL (a pyrogenic silicon dioxide product sold by Cabot Co. of Boston, Mass.) or mixtures thereof.

Surfactants are used in dosage forms to improve the wetting characteristics and/or to enhance dissolution, and are particularly useful in pharmaceutical compositions or dosage forms containing poorly soluble or insoluble drug(s) or active ingredients. Examples of surfactants include, but are not limited to, polyoxyethylene sorbitan fatty acid esters, such as those commercially available as TWEENs (e.g. Tween 20 and Tween 80), polyethylene glycols, polyoxyethylene stearates, polyvinyl alcohol, polyvinylpyrrolidone, poly(oxyethylene)/poly(oxypropylene) block co-polyers such as poloxamers (e.g., commercially available as PLURONICs), and tetrafunctional block copolymers derived from sequential addition of propylene oxide and ethylene oxide to ethylenediamine, such as polyxamines (e.g., commercially as TETRONICs (BASF)), dextran, lecithin, dialkylesters of sodium sulfosuccinic acid, such as Aerosol OT, sodium lauryl sulfate, alkyl aryl polyether sulfonates or alcohols, such as TRITON X-200 or tyloxapol, p-isononylphenoxypoly (glycidol) (e.g. Olin-10G or Surfactant 110-G (Olin Chemicals), or mixtures thereof. Other pharmaceutically acceptable surfactants are well known in the art, and are described in detail in the Handbook of Pharmaceutical Excipients.

Other classes of additives for use with the pharmaceutical compositions or dosage forms of the present invention include, but are not limited to, anti-caking or antiadherent agents, antimicrobial preservatives, coating agents, colorants, desiccants, flavors and perfumes, plasticizers, viscosity increasing agents, sweeteners, buffering agents, humectants and the like.

Examples of anti-caking agents include, but are not limited to, calcium silicate, magnesium silicate, silicon dioxide, colloidal silicon dioxide, talc, or mixtures thereof.

Examples of antimicrobial preservatives include, but are not limited to, benzalkonium chloride solution, benzethonium chloride, benzoic acid, benzyl alcohol, butyl paraben, cetylpyridinium chloride, chlorobutanol, cresol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymol, or mixtures thereof.

Examples of colorants for use with the present invention include, but are not limited to, pharmaceutically acceptable dyes and lakes, caramel, red ferric oxide, yellow ferric oxide or mixtures thereof. Examples of desiccants include, but are not limited to, calcium chloride, calcium sulfate, silica gel or mixtures thereof.

Flavors that may be used include, but are not limited to, acacia, tragacanth, almond oil, anethole, anise oil, benzaldehyde, caraway, caraway oil, cardamom oil, cardamom seed, compound cardamom tincture, cherry juice, cinnamon, cinnamon oil, clove oil, cocoa, coriander oil, eriodictyon, eriodictyon fluidextract, ethyl acetate, ethyl vanillin, eucalyptus oil, fennel oil, glycyrrhiza, pure glycyrrhiza extract, glycyrrhiza fluidextract, lavender oil, lemon oil, menthol, methyl salicylate, monosodium glutamate, nutmeg oil, orange flower oil, orange flower water, orange oil, sweet orange peel tincture, compound orange spirit, peppermint, peppermint oil, peppermint spirit, pine needle oil, rose oil, stronger rose water, spearmint, spearmint oil, thymol, tolu balsam tincture, vanilla, vanilla tincture, and vanillin or mixture thereof.

Examples of sweetening agents include, but are not limited to, aspartame, dextrates, mannitol, saccharin, saccharin calcium, saccharin sodium, sorbitol, sorbitol solution, or mixtures thereof.

Exemplary plasticizers for use with the present invention include, but are not limited to, castor oil, diacetylated monoglycerides, diethyl phthalate, glycerin, mono- and di-acetylated monoglycerides, polyethylene glycol, propylene glycol, and triacetin or mixtures thereof. Suitable viscosity increasing agents include, but are not limited to, acacia, agar, alamic acid, aluminum monostearate, bentonite, bentonite magma, carbomer 934, carboxymethylcellulose calcium, carboxymethylcellulose sodium, carboxymethylcellulose sodium 12, carrageenan, cellulose, microcrystalline cellulose, gelatin, guar gum, hydroxyethyl cellulose, hydroxypropyl cellulose, hydroxypropyl methylcellulose (Nos. 2208; 2906; 2910), magnesium aluminum silicate, methylcellulose, pectin, polyvinyl alcohol, povidone, silica gel, colloidal silicon dioxide, sodium alginate, tragacanth and xanthan gum or mixtures thereof.

Buffering agents that may be used in the present invention include, but are not limited to, magnesium hydroxide, aluminum hydroxide and the like, or mixtures thereof. Examples of humectants include, but are not limited to, glycerol, other humectants or mixtures thereof.

The dosage forms of the present invention may further include one or more of the following: (1) dissolution retarding agents, such as paraffin; (2) absorption accelerators, such as quaternary ammonium compounds; (3) wetting agents, such as, for example, cetyl alcohol and glycerol monostearate; (4) absorbents, such as kaolin and bentonite clay; (5) antioxidants, such as water soluble antioxidants (e.g., ascorbic acid, cysteine hydrochloride, sodium bisulfate, sodium metabisulfate, sodium sulfite and the like), oil soluble antioxidants (e.g., ascorbyl palmitate, hydroxyanisole (BHA), butylated hydroxy toluene (BHT), lecithin, propyl gallate, alpha-tocopherol and the like); and (6) metal chelating agents, such as citric acid, ethylenediamine tetracetic acid (EDTA), sorbitol, tartaric acid, phosphoric acid and the like.

Dosage forms of the present invention, such as a tablet or caplet, may optionally be coated. Inert coating agents typically comprise an inert film-forming agent dispersed in a suitable solvent, and may further comprise other pharmaceutically acceptable adjuvants, such as colorants and plasticizers. Suitable inert coating agents, and methods for coating, are well known in the art, including without limitation aqueous or non-aqueous film coating techniques or microencapsulation. Examples of film-forming or coating agents include, but are not limited to, gelatin, pharmaceutical glaze, shellac, sucrose, titanium dioxide, carnauba wax, microcrystalline wax, celluloses, such as methylcellulose, hydroxymethyl cellulose, carboxymethycellulose, cellulose acetate phthalate, hydroxypropyl methylcellulose (e.g., Nos.: 2208, 2906, 2910), hydroxypropyl cellulose, hydroxypropyl methyl cellulose phthalate (e.g., Nos.: 200731, 220824), hydroxyethylcellulose, methylhydroxyethylcellulose, ethylcellulose which may optionally be cross-linked, and sodium carboxymethyl cellulose; vinyls, such as polyvinyl pyrrolidione, polyvinyl acetate phthalate; glycols, such as polyethylene glycols; acrylics, such as dimethylaminoethyl methacrylate-methacrylate acid ester copolymer, and ethylacrylate-methylmethacrylate copolymer; and other carbohydrate polymers, such as maltodextrins, and polydextrose, or mixtures thereof. The amount of coating agent and the carrier vehicle (aqueous or non-aqueous) used varies upon the type of formulation and mode of administration, and is readily discernible to those of ordinary skill in the art.

A coating of a film forming polymer may optionally be applied to a tablet or caplet (e.g., a capsule shaped tablet) in accordance with the present invention by using one of several types of equipment such as a conventional coating pan, Accelacota, High-Cola or Worster air suspension column. Such equipment typically has an exhaust-system to remove dust and solvent or water vapors to facilitate quick drying. Spray guns or other suitable atomizing equipment may be introduced into the coating pans to provide spray patterns conducive to rapid and uniform coverage of the tablet bed. Normally, heated or cold drying air is introduced over the tablet bed in a continuous or alternate fashion with a spray cycle to expedite drying of the film coating solution.

The coating solution may be sprayed by using positive pneumatic displacement or peristaltic pump systems in a continuous or intermittent spray-dry cycle. The particular type of spray application is selected depending upon the drying efficiency of the coating pan. In most cases, the coating material is sprayed until the tablets are uniformly coated to the desired thickness and the desired appearance of the tablet is achieved. Many different types of coatings may be applied such as enteric, slow release coatings or rapidly dissolving type coatings for fast acting tablets. Preferably, rapidly dissolving type coatings are used to permit more rapid release of the active ingredients, resulting in hastened onset. The thickness of the coating of the film forming polymer applied to a tablet, for example, may vary. However, it is preferred that the thickness simulate the appearance, feel (tactile and mouth feel) and function of a gelatin capsule. Where more rapid or delayed release of the therapeutic agent(s) is desired, one skilled in the art would easily recognize the film type and thickness, if any, to use based on characteristics such as desired blood levels of active ingredient, rate of release, solubility of active ingredient, and desired performance of the dosage form.

A number of suitable film forming agents for use in coating a final dosage form, such as tablets include, for example, methylcellulose, hydroxypropyl methyl cellulose (PHARMACOAT 606 6 cps), polyvinylpyrrolidone (povidone), ethylcellulose (ETHOCEL 10 cps), various derivatives of methacrylic acids and methacrylic acid esters, cellulose acetate phthalate or mixtures thereof.

The method of preparation and the excipients or additives to be incorporated into dosage form (such as a tablet or caplet) are selected in order to give the tablet formulation the desirable physical characteristics while allowing for ease of manufacture (e.g., the rapid compression of tablets). After manufacture, the dose form preferably should have a number of additional attributes, for example, for tablets, such attributes include appearance, hardness, disintegration ability and uniformity, which are influenced both by the method of preparation and by the additives present in the tablet formulation.

Further, it is noted that tablets or other dosage forms of the pharmaceutical compositions of the invention should retain their original size, shape, weight and color under normal handling and storage conditions throughout their shelf life. Thus, for example, excessive powder or solid particles at the bottom of the container, cracks or chips on the face of a tablet, or appearance of crystals on the surface of tablets or on container walls are indicative of physical instability of uncoated tablets. Hence, the effect of mild, uniform and reproducible shaking and tumbling of tablets should be undertaken to insure that the tablets have sufficient physical stability. Tablet hardness can be determined by commercially available hardness testers. In addition, the in vitro availability of the active ingredients should not change appreciably with time.

The tablets, and other dosage forms of the pharmaceutical compositions of the present invention, such as dragees, capsules, pills and granules, may optionally be scored or prepared with coatings and shells, such as enteric coatings and other coatings well known in the pharmaceutical formulating art.

4.3.2 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients (i.e., the compounds of this invention) disclosed herein can also be incorporated into the parenteral dosage forms of the invention.

4.3.3 Transdermal, Topical and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied.

Depending on the specific tissue to be treated, additional components may be used prior to, in conjunction with, or subsequent to treatment with active ingredients of the invention. For example, penetration enhancers can be used to assist in delivering the active ingredients to the tissue.

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

4.3.4 Compositions with Enhanced Stability

The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients.

4.3.5 Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the compounds of this invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

4.3.6 Kits

In some cases, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a single unit dosage form of the compounds of this invention, or a pharmaceutically acceptable salt, prodrug, solvate, hydrate, clathrate or stereoisomer thereof, and a single unit dosage form of another agent that may be used in combination with the compounds of this invention. Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The invention is further defined by reference to the following non-limiting examples. It will be apparent to those skilled in the art that many modifications, both to materials and methods, can be practiced without departing from the spirit and scope of this invention.

### 5. EXAMPLES

### 5.1 Synthesis of Racemic DIDESMETHYLSIBUTRAMINE

An exemplary method of preparing racemic didesmethylsibutramine free base ((R/S)-DDMS) is shown in Scheme 1 below and described in detail below.

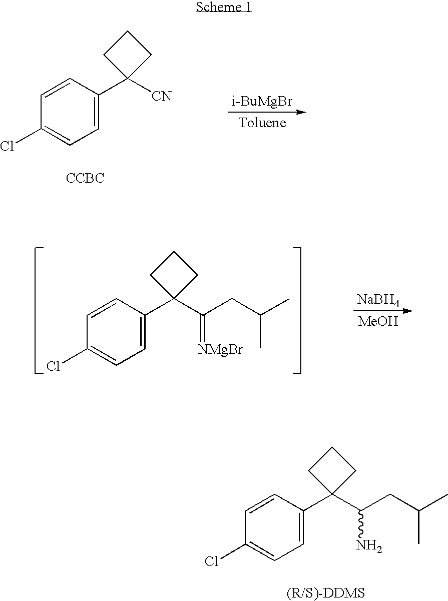

Following Scheme 1, a 1 L three-necked round bottom flask was charged with isobutyl magnesium bromide (200 ml, 2.0 M in diethyl ether) and toluene (159 ml), and the resulting mixture was distilled to remove most of the ether. After the mixture was cooled to 20° C., CCBC (50.0 g) in toluene (45 ml) was added, and the resulting mixture was refluxed for 2-4 hours. The reaction mixture was then cooled to 0° C. and methanol (300 ml) was added to it, followed slowly by NaBH₄ (11 g). The resulting mixture was then added slowly to an aqueous HCL solution (365 ml, 2N) kept at 0° C., and the resulting mixture was warmed to room temperature with continual stirring. After separation of the organic phase, the aqueous phase was washed with toluene (200 ml). The combined organic phase were washed with water (200 ml) and concentrated to give (R/S)-DDMS (55 g, 85%). NMR (CDCl₃): ¹H(δ), 0.6-0.8 (m, 1H), 0.8-1.0 (m, 6H), 1.1-1.3 (m, 1H), 1.6-2.6 (m, 7H), 3.0-3.3 (m, 1H), 7.0-7.6 (m, 4H). ¹³C(δ): 15.4, 21.5, 24.3, 24.7, 31.5, 31.9, 41.1, 50.73, 56.3, 127.7, 129, 131.6, 144.3.

### 5.2 Synthesis of Racemic DIDESMETHYLSIBUTRAMINE.(D)-TARTRATE

An exemplary method of preparing the (D)-tartrate salt of racemic didesmethylsibutramine ((R/S)-DDMS.(D)-TA) is shown below in Scheme 2. The (L)-tartrate salt of racemic didesmethylsibutramine ((R/S)-DDMS.(L)-TA) can be prepared in an analogous manner.

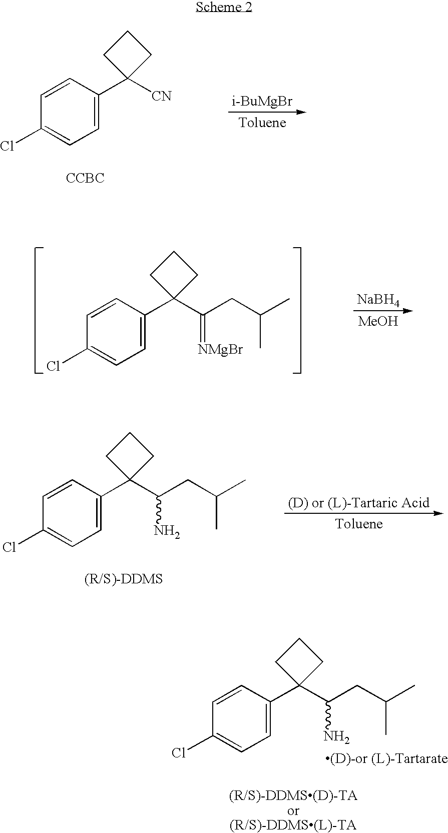

Following Scheme 2, a mixture of racemic didesmethylsibutramine (15.3 g) and toluene (160 ml) was heated to 70-80° C. and (D)-tartaric acid (9.1 g) in water (20 ml) and acetone (10 ml) was added slowly. The resulting mixture was refluxed for 30 minutes, after which the water and acetone were removed by distillation. The resulting mixture was cooled to room temperature to provide a slurry which was then filtered. The resulting wet cake was washed two times with MTBE (20 ml×2) and dried to yield (R/S)-DDMS.(D)-TA (22.5 g, 98%). NMR (DMSO-d₆): ¹H(δ), 0.6-0.92 (m, 6H), 0.92-1.1 (m, 1H), 1.1-1.3 (m, 1H), 1.5-1.8 (m, 2H), 1.8-2.1 (m, 1H), 2.1-2.4 (m, 3H), 2.4-2.6 (m, 1H), 3.4-3.6 (m, 1H), 3.9-4.2 (s, 2H), 6.4-7.2 (b, 6H, OH, COOH and NH₂), 7.3-7.6 (m, 4H). ¹³C(δ): 15.5, 21.1, 23.3, 23.7, 31.5, 37.7, 39.7, 54.5, 72.1, 128, 129.7, 131.3, 142.2, 174.6.

### 5.3 Resolution of (S)-DIDESMETHYLSIBUTRAMINE-(L)-TARTRATE

A method of isolating the (L)-tartrate salt of (S)-didesmethylsibutramine ((S)-DDMS.(L)-TA) from racemic didesmethylsibutramine free base is shown in Scheme 3 and described in detail below.

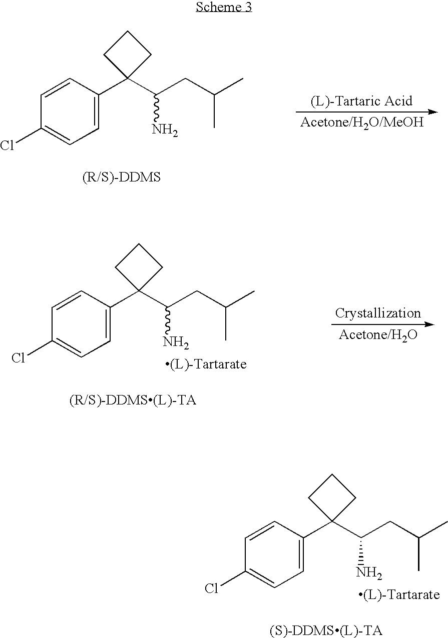

### 5.3.1 Formation of (L)-Tartrate Salt of (S)-DDMS

(R/S)-DDMS (20.5 g), acetone/water/methanol (350 ml, 1:0.13:0.7, v:v:v) and (L)-tartaric acid (12.2 g) were added to a 500 ml three-necked round bottom flask. The mixture was heated to reflux for 30 minutes and then cooled to 45° C. The reaction mixture was then seeded with (S)-DDMS.(L)-TA (10 mg and 99.7% ee) and stirred at 40-45° C. for 30 minutes. The mixture was cooled to room temperature and stirred for 1 hour. The resulting slurry was filtered to provide a wet cake, which was washed with cold acetone/water and dried to give 10.8 g (33.4%) of (S)-DDMS-(L)-TA (89.7% ee).

### 5.3.2 Preparation of (L)-Tartate Salt of (S)-DDMS from Mother Liquor of (R)-DDMS.(D)-TA

A solution of DDMS tartrate in acetone/water/methanol (mother liquor of (R)-DDMS (D)-TA) was concentrated to remove acetone and methanol. The residue was treated with aqueous NaOH (3N, 150 ml) and extracted with ethyl acetate. The organic phase was washed with water (100 ml) and concentrated to give didesmethylsibutramine free base (45 g, 0.18 mol and 36% ee of(S)-isomer). The free amine was charged with (L)-tartric acid (53.6 g, 0.35 mol), acetone (600 ml), water (80 ml), and methanol (40 ml). The mixture was heated to reflux for 1 hour and then cooled to room temperature. The resulting slurry was filtered to provide a wet cake, which was then washed with cold acetone/water two times to give 26.7 g (56% based on (S)-didesmethylsibutramine) of (S)-DDMS.(L)-TA (96% ee).

### 5.3.3 Enrichment of (S)-DDMS.(L)-TA

A mixture of (S)-DDMS.(L)-TA (26.7 g) in acetonitrile/water (475 ml, 1:0.2, v:v) was refluxed for 1 hour and then cooled to room temperature. The resulting slurry was filtered and dried to give 17.4 g (65%) of (S)-DDMS.(L)-TA (99.9% ee; 99.94% chemical purity). NMR (DMSO-d₆): ¹H (6), 0.7-0.9 (m, 6H), 0.9-1.05 (m, 1H), 1.1-1.3 (b, 1H), 1.52-1.8 (b, 2H), 1.84-2.05 (b, 1H), 2.15-2.4 (b, 3H), 2.4-2.6 (b, 1H), 3.65-3.68 (m, 1H), 4.0 (s, 2H), 6.7-7.3 (b, 6H from NH₂, OH and COOH), 7.1-7.6 (m, 4H). 13C(δ): 15.4, 21.5, 22.0, 22.2, 32.0, 32.2, 38.4, 49.0, 54.0, 72.8, 128.8, 130.0, 132.0, 143.0, 175.5.

5.4 Determination of Potency and Specificity

A pharmacologic study is conducted to determine the relative potency, comparative efficacy, binding affinity and toxicity of (S)-didesmethylsibutramine, or a pharmaceutically acceptable salt, hydrate, solvate, clathrate or prodrug thereof. The profile of relative specificity of monoamine reuptake inhibition is determined from the compounds' inhibition of norephinephrine (NE) reuptake in brain tissue with that of the inhibition of dopamine (DA) and serotonin (5-HT) reuptake.

High-affinity uptake of the ³H-radiomonoamines is studied in synaptosomal preparations prepared from rat corpus striatum (for inhibition of DA reuptake) and cerebral cortex (for 5-HT and NE) using methods published by Kula et al., Life Sciences, 34(26): 2567-2575 (1984) and Baldessarini et al., Life Sciences, 39: 1765-1777 (1986), both of which are incorporated herein by reference. Tissues are freshly dissected on ice and weighed. Following homogenization by hand (14 strokes in 10-35 volumes of ice-cold isotonic 0.32 M sucrose, containing nialamide, 34 μM) in a Teflon-on-glass homogenizer, the tissue is centrifuged for ten minutes at 900×g; the supernatant “solution” that results contains synaptosomes that are used without further treatment. Each assay tube contains 50 μL of the cerebral homogenate, radio-labeled ³H-monoamine, and the test compound, e.g., (S)-didesmethylsibutramine, in a freshly prepared physiologic buffer solution with a final volume of 0.5 ml.

Tissues are preincubated for 15 minutes at 37° C. before the assay. Tubes are held on ice until the start of incubation, which is initiated by adding ³H-amine to provide a final concentration of 0.1 μM. Tubes are incubated at 37° C. for 10 minutes with ³H-DA (26 Ci/mmol) and for 20 minutes with ³H-5-HT (about 20 Ci/mmol) and ³H-NE (about 20 Ci/mmol). The specific activity of the radiomonoamine will vary with available material and is not critical. The reaction is terminated by immersion in ice and dilution with 3 ml of ice cold isotonic saline solution containing 20 mM TRIS buffer (pH 7.0). These solutions are filtered through cellulose ester microfilters, followed by washing with two 3 ml volumes of the same buffer. The filter is then counted for ³H-radioactivity in 3.5 ml of Polyfluor at about 50% efficiency for tritium. Blanks (either incubated at 0° C. or incubated with specific, known uptake inhibitors of DA (e.g., GRB-12909, 10 μM), 5-HT (e.g., zimelidine, 10 μM) or NE (e.g., desipramine, 10 μM)) are usually distinguishable from assays performed without tissue and average 2-3% of total CPM.

Comparison of the amounts of 3H-radioactivity retained on the filters provides an indication of the relative abilities of enantiomerically pure (S)-didesmethylsibutramine and of known DA, 5-HT and NE reuptake inhibitors to block the reuptake of these monoamines in those tissues. This information is useful in gauging the relative potency and efficacy of compounds of the invention.

The acute toxicities of the compounds of this invention are determined in studies in which rats are administered progressively higher doses (mg/kg) of the compounds of this invention. The lethal dose, which, when administered orally, causes death of 50% of the test animals, is reported as the LD₅₀. Comparison of LD₅₀ values for the compounds of this invention and other compounds provides a measure of the relative toxicity of the compositions.

5.4.1 Muscarinic, 5-HT, and NE Binding Affinities

The binding affinities of racemic sibutramine ((R/S)-sibutramine), and racemic and enantiomerically pure didesmethylsibutramine ((R/S)-, (R)-, and (S)-DDMS) were determined at the nonselective muscarinic receptor and the serotonin (5-HT) uptake site from rat cerebral cortex, and the human recombinant norepinephrine (NE) uptake site. Compounds were tested initially at 10 μM in duplicate, and if ≧50% inhibition of specific binding was observed, they were tested further at 10 different concentrations in duplicate to obtain full competition curves. IC₅₀ values (concentration required to inhibit 50% specific binding) were then determined by nonlinear regression analysis of the curves and tabulated below.

| | Binding IC₅₀ | | |
|---|---|---|---|
| | Values (nM) | | |
| | Mucarinic | NE | 5-HT |
| Compound | Receptor | Uptake | Uptake |
| (R/S)-Sibutramine | 2,650 | 350 | 2,800 |
| (R/S)-DDMS | — | 16 | 63/14 |
| (R)-DDMS | — | 13 | 140 |
| (S)-DDMS | — | 6.2 | 4,3000 |
| Atropine | 0.31 | — | — |
| Imipramine | — | — | 145/32 |
| Zimelidine | — | — | 129 |
| Protriptyline | — | 3.6/0.9 | — |

Affinity for the muscarinic site was weak for all compounds compared to atropine, and binding to the 5-HT and NE uptake sites was orders of magnitude less than that of the standards.

The above data, which was generated as described above, show that (S)-didesmethylsibutramine is a potent inhibitor of NE uptake without appreciable 5-HT or muscarinic activity.

The skilled artisan will readily understand that various additional in vitro or in vivo studies can be performed, such as other receptor binding studies or functional monoamine uptake assays, including without limitation testing for inhibition of functional uptake of various compounds, such as serotonin (5-HT), norepinephrine (NE), and dopamine (DA), in human recombinant monoamine transporters expressed in various cell types, e.g., HEK-293hSERT cells (for 5-HT), MDCK dog kidney cells (for NE), and CHO-Ki/hDAT cells (for DA).

5.5 Oral Formulation

Hard gelatin capsule dosage forms that are lactose-free comprising (S)-didesmethylsibutramine, or a pharmaceutically acceptable salt, hydrate, solvate, clathrate or prodrug thereof, can be prepared using the following ingredients:

| | 5 mg | 10 mg | 20 mg |
|---|---|---|---|
| Component | Capsule | Capsule | Capsule |
| (S)- | 5.0 | 10.0 | 20.0 |
| didesmethylsibutramine | | | |
| Microcrystalline | 90.0 | 90.0 | 90.0 |
| Cellulose | | | |
| Pre-gelatinized Starch | 100.3 | 97.8 | 82.8 |
| Croscarmellose | 7.0 | 7.0 | 7.0 |
| Magnesium Stearate | 0.2 | 0.2 | 0.2 |

The enantiomerically pure (S)-didesmethylsiburamine is sieved and blended with the excipients listed. The mixture is filled into suitably sized two-piece hard gelatin capsules using suitable machinery and methods well known in the art. See, e.g., Remington's Pharmaceutical Sciences, 16^{th or} 18^{th} Edition, each of which is incorporated herein in its entirety by reference. Other doses can be prepared by altering the fill weight and, if necessary, changing the capsule size to suit. Any of the stable, non-lactose hard gelatin capsule formulations above can be formed.

Compressed tablet dosage forms of (S)-didesmethylsibutramine, or a pharmaceutically acceptable salt, hydrate, solvate, clathrate or prodrug thereof, can be prepared using the following ingredients:

| | 5 mg | 10 mg | 20 mg |
|---|---|---|---|
| Component | Capsule | Capsule | Capsule |
| (S)- | 5.0 | 10.0 | 20.0 |
| didesmethylsibutramine | | | |
| Microcrystalline | 90.0 | 90.0 | 90.0 |
| Cellulose | | | |
| Pre-gelatinized Starch | 100.3 | 97.8 | 82.8 |
| Croscarmellose | 7.0 | 7.0 | 7.0 |
| Magnesium Stearate | 0.2 | 0.2 | 0.2 |

The enantiomerically pure (S)-didesmethylsibutramine is sieved through a suitable sieve and blended with the non-lactose excipients until a uniform blend is formed. The dry blend is screened and blended with the magnesium stearate. The resulting powder blend is then compressed into tablets of desired shape and size. Tablets of other strengths can be prepared by altering the ratio of the active ingredient to the excipients or modifying the tablet weight.

The embodiments of the invention described above are intended to be merely exemplary and those skilled in the art will recognize, or be able to ascertain using no more than routine experimentation, numerous equivalents to the specific procedures described herein. All such equivalents are considered to be within the scope of the invention and are encompassed by the following claims.

All of the patents, patent applications and publications referred to in this application are incorporated herein in their entireties. Moreover, citation or identification of any reference in this application is not an admission that such reference is available as prior art to this invention. The full scope of the invention is better understood with reference to the appended claims.

1. A method of treating, preventing or managing a sleep disorder comprising administering to a patient in need of such treatment, prevention or management a therapeutically or prophylactically effective amount of enantiomerically pure (S)-didesmethylsibutramine, or a pharmaceutically acceptable salt, hydrate, solvate, clathrate, or prodrug thereof.

2. The method of claim 1, wherein the sleep disorder is insomnia.

3. The method of claim 2, wherein the insomnia is complete wakefulness.

4. The method of claim 1, wherein the sleep disorder is a circadian rhythm sleep disorder.

5. The method of claim 1, wherein the sleep disorder is periodic limb movement disorder.

6. The method of claim 4, wherein the circadian rhythm sleep disorder is shift work sleep disorder.

7. The method of claim 1, wherein the (S)-didesmethylsibutramine comprises greater than about 90 percent by weight of didesmethylsibutramine.

8. The method of claim 7, wherein the (S)-didesmethylsibutramine comprises greater than about 95 percent by weight of didesmethylsibutramine.

9. The method of claim 8, wherein the (S)-didesmethylsibutramine comprises greater than about 97 percent by weight of didesmethylsibutramine.

10. The method of claim 9, wherein the (S)-didesmethylsibutramine comprises greater than about 99 percent by weight of didesmethylsibutramine.

11. The method of claim 1, wherein the amount of (S)-didesmethylsibutramine administered is from about 0.1 mg to about 60 mg per day.

12. The method of claim 11, wherein the amount of (S)-didesmethylsibutramine administered is from about 2 mg to about 30 mg per day.

13. The method of claim 12, wherein the amount of (S)-didesmethylsibutramine administered is from about 5 mg to about 15 mg per day.

14. The method of claim 1, wherein the (S)-didesmethylsibutramine is administered by oral, mucosal, rectal or parenteral administration.

15. The method of claim 14, wherein the (S)-didesmethylsibutramine is administered by oral administration.

16. The method of claim 14, wherein the (S)-didesmethylsibutramine is administered by parenteral administration.

17. The method of claim 16, wherein the (S)-didesmethylsibutramine is administered by intravenous, intramuscular or subcutaneous administration.

18. A method of treating or preventing insomnia comprising administering to a patient in need of such treatment or prevention a therapeutically or prophylactically effective amount of enantiomerically pure (S)-didesmethylsibutramine, or a pharmaceutically acceptable salt, hydrate, solvate, clathrate, or prodrug thereof.

19. A method of managing insomnia comprising administering to a patient in need of such management a therapeutically effective amount of enantiomerically pure (S)-didesmethylsibutramine, or a pharmaceutically acceptable salt, hydrate, solvate, clathrate, or prodrug thereof.

20. The method of claims 18 or 19, wherein the insomnia is transient insomnia, chronic insomnia, initial insomnia, middle insomnia, or terminal insomnia.

21. The method of claims 18 or 19, wherein the insomnia is primary insomnia or secondary insomnia.

22. A method of treating or preventing complete wakefulness comprising administering to a patient in need of such treatment or prevention a therapeutically or prophylactically effective amount of enantiomerically pure (S)-didesmethylsibutramine, or a pharmaceutically acceptable salt, hydrate, solvate, clathrate, or prodrug thereof.

23. A method of managing complete wakefulness comprising administering to a patient in need of such management a therapeutically effective amount of enantiomerically pure (S)-didesmethylsibutramine, or a pharmaceutically acceptable salt, hydrate, solvate, clathrate, or prodrug thereof.

24. A method of treating or preventing shift work sleep disorder comprising administering to a patient in need of such treatment or prevention a therapeutically or prophylactically effective amount of enantiomerically pure (S)-didesmethylsibutramine, or a pharmaceutically acceptable salt, hydrate, solvate, clathrate, or prodrug thereof.

25. A method of managing shift work sleep disorder comprising administering to a patient in need of such management a therapeutically effective amount of enantiomerically pure (S)-didesmethylsibutramine, or a pharmaceutically acceptable salt, hydrate, solvate, clathrate, or prodrug thereof.

26. A method of treating or preventing periodic limb movement disorder comprising administering to a patient in need of such treatment or prevention a therapeutically or prophylactically effective amount of enantiomerically pure (S)-didesmethylsibutramine, or a pharmaceutically acceptable salt, hydrate, solvate, clathrate, or prodrug thereof.

27. A method of managing periodic limb movement disorder comprising administering to a patient in need of such management a therapeutically effective amount of enantiomerically pure (S)-didesmethylsibutramine, or a pharmaceutically acceptable salt, hydrate, solvate, clathrate, or prodrug thereof.
